SEQUENCE LISTING (9 sequences)

SEQ ID NO: 1 - DNA, 27 bp, Artificial Sequence
  Primer
agctctcgagcggcaagccacagtcag

SEQ ID NO: 2 - DNA, 28 bp, Artificial Sequence
  Primer
aaatgcggccgcttctccgttggggtct

SEQ ID NO: 3 - DNA, 32 bp, Artificial Sequence
  Primer
acagcggccgcaaatgggctcttcaggtgttg

SEQ ID NO: 4 - DNA, 30 bp, Artificial Sequence
  Primer
gcggggatccacacgtcagtcccctagcca

SEQ ID NO: 5 - DNA, 29 bp, Artificial Sequence
  Primer
cttatctagataaggtaccgggccctact

SEQ ID NO: 6 - protein, 28 aa, Artificial Sequence
  Sequence comprising a FLAG-tag
CAAVITSEFDIKLIDTVDLEDYKDDDDK

SEQ ID NO: 7 - protein, 545 aa, Rattus rattus
MALPSPASEEAEGPCQEANQEYQEPVCSPVPEPEPEPEPEPEPEPEPVPVPPPEPQPEPEPQPLPDPAPLPVLGFEAEPVQEPEPTPTVETRGTARGFQPPEGGFGWIVVFAATWCNGSIFGIHNSVGILYSMLLEEEKEKNRQVEFQAAWVGALAMGMIFFCSPIVSTFIDRLGCRITATTGAAVAFIGLHTSSFTSPLSLRYFTYGILFGCGCSFAFQPSLVILDHYFQRRLGLANGVVSAGSSIFSMSFPFLIKMLGDRIKLAQTFQVLSTFMFVLTLLSLTYRPLLPSSQDTPSKRGAHTLRQRFLVQFRKYFNMRVFRQRTYRIWAFGIAAAALGYFVPYVHLMKYVEDKFKEIKETWVLLVCIGATSGLGRLVSGHISDSIPGLKKIYLQVLSFLLLGLMSMMIPLCRDFGGLIVVCLFLGLCDGFFITIMAPIAFELVGPMQASQAIGYLLGMMALPMIAGPPIAGLLRNCFGNYHVAFYFAGVPPIIGAVILFFVPLMHQRMFKKEQRESSKDKMLSHDPDPNGELLPGSPTPEEPI

SEQ ID NO: 8 - protein, 565 aa, Mus musculus
MALPSPASEEAEGPCQEANQEYQEPVCSPVPEPEPEPEPEPEPDPEPVPVPPPEPQPEPEPQPLPDPAPLPELGFEAEPEPQPLPDPAPLPELGFEAEPVQEPEPTPTVETRGTARGFQPPEGGFGWIVVFAATWCNGSIFGIHNSVGILYSMLLEEEKEKNRQVEFQAAWVGALAMGMIFFCSPIVSIFTDRLGCRITATTGAAVAFIGLHTSSFTSSLSLRYFTYGILFGCGCSFAFQPSLVILDHYFQRRLGLANGVVSAGSSIFSMSFPFLIKMLGDKIKLAQTFQVLSTFMFVLTLLSLTYRPLLPSSQDTPSKRGAHTLRQRFLVQFRKYFNMRVFRQRTYRIWAFGIAAAALGYFVPYVHLMKYVEDKFKEIKETWVLLVCIGATSGLGRLVSGHISDSIPGLKKIYLQVLSFLLLGLMSMMIPLCRDFGGLIVVCLFLGLCDGFFITIMAPIAFELVGPMQASQAIGYLLGMMALPMIAGPPIAGLLRNCFGDYHVAFYFAGVPPIIGAVILFFVPLMHQRMFKKEQRDSSKDKMLSHDPDPNGELLPGSPTPEEPI

SEQ ID NO: 9 - protein, 539 aa, Homo sapiens
MALQSQASEEAKGPWQEADQEQQEPVGSPEPESEPEPEPEPEPVPVPPPEPQPEPQPLPDPAPLPELEFESERVHEPEPTPTVETRGTARGFQPPEGGFGWVVVFAATWCNGSIFGIHNSVGILYSMLLEEEKEKNRQVEFQAAWVGALAMGMIFFCSPIVSIFTDRLGCRITATAGAAVAFIGLHTSSFTSSLSLRYFTYGILFGCGCSFAFQPSLVILGHYFQRRLGLANGVVSAGSSIFSMSFPFLIRMLGDKIKLAQTFQVLSTFMFVLMLLSLTYRPLLPSSQDTPSKRGVRTLHQRFLAQLRKYFNMRVFRQRTYRIWAFGIAAAALGYFVPYVHLMKYVEEEFSEIKETWVLLVCIGATSGLGRLVSGHISDSIPGLKKIYLQVLSFLLLGLMSMMIPLCRDFGGLIVVCLFLGLCDGFFITIMAPIAFELVGPMQASQAIGYLLGMMALPMIAGPPIAGLLRNCFGDYHVAFYFAGVPPIIGAVILFFVPLMHQRMFKKEQRDSSKDKMLAPDPDPNGELLPGSPNPEEPI

(19) United States
(12) Patent Application Publication
(10) Pub. No.: US 2005/0159352 A1
(43) Pub. Date: Jul. 21, 2005

(54) Use of monocarboxylate transporter protein for thyroid hormone transport

(76) Inventors: Edith C. H. Friesema, Poortugaal (NL); Theofilus J. Visser, Poortugaal (NL); Eric P. Krenning, Rotterdam (NL)

Correspondence Address: TRASK BRITT, P.O. BOX 2550, SALT LAKE CITY, UT, 84110, US

(21) Appl. No.: 10/997,757

(22) Filed: Nov. 24, 2004

Related U.S. Application Data
(63) Continuation of application No. PCT/NL03/00384, filed on May 23, 2003.

(30) Foreign Application Priority Data
May 24, 2002 (EP) 02077061.6

(51) Int. Cl. A61K 38/17; A61K 31/198; C07K 14/705

(52) U.S. Cl. 514/12; 514/567; 530/350

(57) ABSTRACT

The use of a monocarboxylate transporter protein or a functional part, derivative and/or analogue thereof for altering transport of a thyroid hormone or a functional part, derivative and/or analogue thereof across a membrane. The monocarboxylate transporter protein preferably comprises MCT-8. An isolated molecule capable of specifically binding at least part of an MCT protein, at least part of a ligand of an MCT protein, or a nucleic acid encoding the MCT and/or ligand, is also herewith provided. Regulation of the bioavailability of thyroid hormone in a tissue enables interfering with diseases. The invention also provides pharmaceutical compositions comprising a compound capable of binding the MCT protein or capable of influencing the binding or transporting of a ligand of the MCT protein. Methods for diagnosis and/or treatment of a disease, such as a disorder of thyroid metabolism, non-thyroidal illness, obesity or cardiovascular illness, are also provided, as well as bioassays for identifying or detecting a candidate drug capable of binding to, or influencing at least part of, the MCT protein.

### CROSS-REFERENCE TO RELATED APPLICATION

This application is a continuation of PCT International Patent Application No. PCT/NL03/00384, filed on May 23, 2003, designating the United States of America, and published, in English, as PCT International Publication No. WO 03/099864 A1 on Dec. 4, 2003, the contents of the entirety of which are incorporated by reference.

### TECHNICAL FIELD

The invention relates to the field of endocrinology, more specifically, to the field of regulating thyroid hormone bioavailability and activity.

### BACKGROUND

Thyroid hormone is essential for the development and homeostasis of different organs, particularly for the regulation of energy metabolism. The follicular cells of the thyroid gland predominantly produce the prohormone thyroxine (3,3′,5,5′-tetraiodothyronine, T4), which has little or no biological activity. T4 is activated by enzymatic outer ring deiodination to 3,3′,5-triiodothyronine (T3), which is the most, if not only, bioactive form of thyroid hormone (1-4). Both T4 and T3 are inactivated by enzymatic inner ring deiodination to the metabolites 3,3′,5′-triiodothyronine (reverse T3, rT3) and 3,3′-diiodothyronine (3,3′-T2), respectively (1-4).

The three deiodinases involved in these processes show different catalytic profiles, tissue distributions and regulatory functions. They have recently been identified as homologous transmembrane selenoproteins with their active site exposed to the cytoplasm (1-4).

Additional pathways of thyroid hormone metabolism include sulfation and glucuronidation of the phenolic hydroxyl group by transferases located in the cytoplasm and endoplasmic reticulum of different tissues (2, 4).

Thyroid hormone is essential for the development of different tissues, for example, the brain, and for the regulation of energy metabolism of a wide range, if not all, of tissues throughout life (5) and regulating its bioavailability or activity in various tissues would enable regulating, influencing or interfering with (metabolic) disease in those tissues.

Considering the central role that is played by thyroid hormone in the basal metabolism of a wide variety of cells in a wide range of tissues, the use of drugs comprising thyroid hormone agonists and/or antagonists will have beneficial effects in many diseases.

Access of plasma thyroid hormone to intracellular receptors and enzymes requires transport across the cell membrane. On the basis of the lipophilic nature of iodothyronines, it was assumed for a long time that they cross the cell membrane by simple diffusion. However, this ignored the highly polar nature of the alanine side chain which is a formidable obstacle for membrane passage of iodothyronines. During the last two decades, although none were found, overwhelming evidence has accumulated indicating the involvement of plasma membrane transporters in tissue uptake of thyroid hormone (4,6).

In EP 0982399 it was reported that such plasma membrane thyroid transporters indeed exist. In that patent application plasma membrane thyroid transporters are provided, such as polypeptides related to an organic anion transporting peptide. These peptides comprise sodium-dependent taurocholate co-transporting polypeptide or sodium-independent organic anion-transporting peptide. An example of such a polypeptide is rat Ntcp, which is a 362-aminoacid protein containing 7 putative transmembrane domains and 2 glycosylation sites with an apparent molecular mass of 51 kDa (7,9,10). It and its orthologues are only expressed in differentiated mammalian hepatocytes, where it is localized selectively to the basolateral cell membrane (9, 10). It is the major transporter of conjugated bile acids in liver but it also mediates uptake of unconjugated bile acids (9, 10 11). The reported Km values of taurocholate for rat Ntcp vary between 15 and 51 jiM (10). Ntcp may also mediate transport of a number of non-bile acid amphipathic compounds, including estrogen conjugates such as estrone 3-sulfate (11).

Another example is rat oatp1 which is a 670-aminoacid protein with 12 transmembrane domains and 2 glycosylation sites with an apparent molecular mass of 80 kDa (8-10). Oatp1 is not only expressed in liver but also in kidney and brain. Like Ntcp, oatp1 is localized to the basolateral liver cell membrane. Oatp1 is a multispecific transporter mediating the uptake of a wide variety of amphipathic ligands (9, 10, 12-15), including conjugated and unconjugated bile acids, conjugated steroids (e.g., estrone sulfate, estradiol 17β-glucuronide and DHEA sulfate) and other organic anions (e.g., the prototypic bromosulfophthalein), but also neutral steroids (e.g. aldosterone and cortisol), cardiac glycosides (e.g., ouabain) and even organic cations (e.g., ajmalinium). Apparent Km values of taurocholate and bromosulfophthalein for rat oatp1 amount to 50 and 1.5 μM, respectively (10). In contrast to Ntcp, transport through oatp1 is not coupled to Na⁺. Various members of the oatp transporter family in humans and rats have now been shown to be capable of transporting thyroid hormone (16).

### DISCLOSURE OF THE INVENTION

The present invention provides alternative plasma membrane thyroid transporters. Surprisingly it has been found by the present inventors that a monocarboxylate transporter protein (MCT) is capable of transporting a thyroid hormone across a plasma membrane. This class of proteins is reported to transport monocarboxylates such as lactate and pyruvate (Reviewed in (17)). Until now, no correlation with thyroid hormone transport was known. The invention provides a use of a monocarboxylate transporter (MOT) protein or a functional part, derivative and/or analogue thereof for altering transport of a thyroid hormone or a functional part, derivative and/or analogue thereof across a membrane. Preferably, the transport is enhanced. When the term “MCT protein” is mentioned in the description, it can also refer to a functional part, derivative and/or analogue of an MCT protein. Likewise, the term “thyroid hormone” as used herein is also meant to comprise a functional part, derivative and/or analogue of the thyroid hormone.

Transport of a thyroid hormone across a membrane, such as for instance a plasma membrane, can for instance be enhanced by providing MCT protein to the membrane. Uptake of the MCT protein in the membrane can increase the concentration of the MCT protein. A higher concentration of MCT can result in increased transport of thyroid hormone across the membrane. An MCT protein can be directly provided to a membrane (preferably a plasma membrane) by administration of the protein to the membrane and/or to a cell comprising the membrane. In one embodiment, essentially pure protein is administered. In another embodiment, the protein is part of a (pharmaceutical) composition. The composition preferably comprises a suitable carrier and/or adjuvant.

An MCT protein can as well be indirectly provided to a (preferably plasma) membrane, for instance by providing a cell comprising the membrane with a nucleic acid encoding the protein. This can for instance be performed by a gene delivery vehicle. Upon expression of the MCT protein by the cell, the amount of MCT protein in the membrane is increased. The increased amount of MCT results in enhanced thyroid hormone transport across the membrane.

Transport of a thyroid hormone can also be altered by a molecule capable of specifically binding a monocarboxylate transporter protein or a functional part, derivative and/or analogue thereof, or a ligand of the monocarboxylate transporter protein. Such isolated molecule is therefore also herewith provided. In one embodiment, the molecule comprises a proteinaceous molecule such as a peptide or polypeptide (“(poly)peptide”). A molecule with specific binding properties can be generated and/or identified by methods known in the art. For instance, one can use Pepscan techniques and/or replacement mapping techniques as well as for instance phage-display techniques and/or screening of combinatorial libraries, allowing identification of active sites in a polypeptide sequence.

A molecule capable of specifically binding at least part of an MCT protein, or capable of specifically binding at least part of a ligand of an MOT protein, can for instance act as an antagonist, decreasing transport of thyroid hormone across a membrane by the MCT protein. Alternatively, the molecule can act as an agonist, enhancing thyroid hormone transport. The invention thus provides a use of a molecule of the invention for altering transport of a thyroid hormone or a functional part, derivative and/or analogue thereof across a membrane. Preferably, a use of the invention is provided wherein the membrane comprises a plasma membrane.

In one embodiment of the invention, transport of a thyroid hormone is altered by a molecule capable of specifically binding a nucleic acid sequence encoding a monocarboxylate transporter protein or a functional part, derivative and/or analogue thereof, and/or encoding a ligand of the monocarboxylate transporter protein or functional part, derivative and/or analogue. The molecule is therefore also herewith provided. The molecule may be administered to a cell comprising the nucleic acid sequence encoding MCT and/or a ligand thereof. The molecule may be administered either directly or indirectly, for instance by administration of a nucleic acid sequence encoding the molecule. Upon administration, the molecule will specifically bind the nucleic acid sequence and, hence, will influence the expression of the MCT and/or the ligand. This results in an altered MCT and/or ligand content of the cell and, as a result, in an altered transport of a thyroid hormone or functional part, derivative and/or analogue thereof. The altered expression of the MCT and/or the ligand preferably results in an enhanced transport of the thyroid hormone or functional part, derivative and/or analogue.

By “a molecule capable of specifically binding a nucleic acid sequence” is meant herein a molecule that is capable of distinguishing between related nucleic acid sequences under stringent conditions. A molecule capable of specifically binding a proteinaceous molecule is defined as a molecule that is capable of distinguishing between different proteinaceous molecules because it has a higher affinity for a specific amino acid sequence of the proteinaceous molecule, a specific conformation of the proteinaceous molecule, etc. Non-specific “sticking” of a molecule is not considered “specific binding”.

Once a molecule of the invention, capable of specifically binding at least part of an MCT protein, or a functional part, derivative, analogue and/or ligand thereof, or a nucleic acid encoding therefore, has been generated and/or isolated, a desired property, such as its binding capacity, can subsequently be improved. In case of a proteinaceous molecule of the invention this can for instance be done by an Ala-scan and/or replacement net mapping method. With these methods, many different proteinaceous molecules are generated, based on an original amino acid sequence but each molecule containing a substitution of at least one amino acid residue. The amino acid residue may either be replaced by alanine (Ala-scan) or by any other amino acid residue (replacement net mapping). Each variant is subsequently screened for the desired property. Generated data are used to design an improved proteinaceous molecule.

In a preferred embodiment, the monocarboxylate transporter protein comprises monocarboxylate transporter protein-8 (MCT8). MCT8 was cloned in 1994 by Lafreniere et al. but its role had never been elucidated. The gene consists of 6 exons coding for a protein with 12 putative transmembrane domains. MCT8 is highly expressed in liver, but also in heart, brain, placenta, lung and kidney. As is illustrated by the examples, MCT-8 shows very good thyroid hormone transporter activity. Transport of thyroid hormone across a plasma membrane appears even to be carried out more efficiently by MCT-8 as compared to currently known Ntcp and oatp transporters. However, other members of the MCT family such as MCT-1, MOT-2, MCT-3, MCT-4, MCT-5, MCT-6, MCT-7 and MCT-9 are also within the scope of the present invention.

A functional part of a protein is defined as a part which has the same kind of properties in kind, not necessarily in amount. A functional derivative of a protein is defined as a protein which has been altered such that the properties of the molecule are essentially the same in kind, not necessarily in amount. A derivative can be provided in many ways, for instance through conservative amino acid substitution.

A person skilled in the art is well able to generate analogous compounds of a protein. This can for instance be done through screening of a peptide library. Such an analogue has essentially the same properties of the protein in kind, not necessarily in amount.

A functional part of a thyroid hormone is defined as a part which has the same kind of properties of influencing development of a tissue and/or regulating energy metabolism of a tissue in kind, not necessarily in amount. A functional derivative of a thyroid hormone is defined as a proteinaceous molecule which has been altered such that the properties of the molecule are essentially the same in kind, not necessarily in amount. A derivative can be provided in many ways, for instance through conservative amino acid substitution. An analogue of a thyroid hormone has essentially the same properties of a thyroid hormone in kind, not necessarily in amount. The analogue preferably comprises a prohormone thyroxine 3,3′,5,5′-tetraiodothyronine (T4) or a molecule that is derived from the prohormone. Preferably, the molecule is derived from the prohormone by outer and/or inner ring deiodination. In one embodiment the analogue comprises 3,3′,5′-triiodothyronine (reverse T3) and/or 3,3′-diiodothyronine (3,3′-T2). As used herein, 3,3′-T2 is also referred to as T2.

In one aspect the invention provides a compound capable of influencing the binding or transporting of a ligand of, or capable of binding to, a plasma membrane polypeptide capable of transporting a thyroid hormone, wherein the polypeptide comprises a monocarboxylate transporter protein or a functional part, derivative and/or analogue thereof, for use as a medicament. In another aspect the invention provides a compound capable of specifically binding a nucleic acid sequence encoding a monocarboxylate transporter protein or a functional part, derivative and/or analogue thereof, or encoding a ligand of the monocarboxylate transporter protein, for use as a medicament.

Preferably, the polypeptide is capable of transporting 3,3′,5,5′-tetraiodothyronine, 3,3′,5-triiodothyronine, 3,3′,5′-truodothyronine and/or 3,3′-diiodothyronine. More preferably, the polypeptide is capable of transporting 3,3′,5-triiodothyronine. In another preferred embodiment the polypeptide is capable of transporting 3,3′,5,5′-tetraiodothyronine. In a most preferred embodiment, the monocarboxylate transporter protein comprises monocarboxylate transporter protein-S.

A compound as provided by the invention is suitable for use in methods for treating a wide range of disorders, such as disorders of thyroid hormone metabolism, for example related to brain disorders seen with psychiatric disease, to restore or help develop tissue metabolism or development in premature children, to help restore thyroid hormone function in patients wherein the thyroid has been removed or is dysfunctioning, for example due to a malignancy, to help alleviate non-thyroidal illness, to treat obesity or cardiovascular illness, to name a few. In fact, all disorders or diseases wherein the (basal) metabolism of a cell or tissue is affected can be treated with a compound of the invention, which can act as an agonist or as an antagonist. An agonist mainly acts in up-regulating metabolism and an antagonist mainly acts in down-regulating metabolism. In a preferred embodiment, such a compound of the invention comprises a peptide, preferably a synthetic peptide, or an antibody or other binding molecule or thyroid hormone analogue capable of binding to or influencing or interfering with the binding or transporting of a ligand (for example T3) of MCT. A compound of the invention is preferably capable of binding at least part of MCT, and/or at least part of a ligand of MCT. Optionally a compound as provided by the invention is provided with a carrier known in the art for production of a medicament. The carrier may be a diluent.

Compounds comprising tissue specific thyroid hormone agonists and/or antagonists as provided by the invention can for example be used in treating obesity, heart failure or (tissue specific) hypo- or hyper-thyroidism. Another example is when tissue specific malignancies such as tumors require up- or down-regulation. Such malignancies can be treated by determining which thyroid hormone transporter is used by the cells in the tissue or malignancy, (for example by biopsy and histochemistry using immunological detection or detection of mRNA expression) and then treating the patient with an agonist/antagonist as provided by the invention which specifically acts through the then determined transporter. Furthermore, compounds comprising (tissue specific) thyroid hormone agonists/antagonists as provided by the invention can be used for the production of a medicament for treating a disorder of thyroid metabolism, non-thyroidal illness, obesity or cardiovascular illness. Thus, the invention provides a use of a compound of the invention for the production of a medicament for the treatment of a thyroid hormone related disorder, non-thyroidal illness, obesity or cardiovascular illness.

A pharmaceutical composition comprising a compound of the invention and a suitable carrier is also herewith provided. The carrier preferably comprises a suitable adjuvant such as for instance Specol or a double oil emulsion. Moreover, the compound may be coupled to a solid carrier, such as keyhole limpet hemocyanin (KLH) or an immunogenic conjugate of a protein such as ovalbumin. Such pharmaceutical compound is particularly suitable for treating a disorder such as a disorder of thyroid metabolism, non-thyroidal illness, obesity or cardiovascular illness. In one embodiment the invention therefore provides a method for treating a disorder such as a disorder of thyroid metabolism, non-thyroidal illness, obesity or cardiovascular illness, comprising administering a compound or a pharmaceutical composition of the invention to an individual suffering from the disorder. The compound or pharmaceutical composition can for instance be administered to an individual orally, by aerosol or as a suppository. Alternatively, the compound can be administered with aid of gene therapy, involving administration of a nucleic acid encoding at least part of the compound, and expression and translation of the nucleic acid, preferably in a host cell. The nucleic acid for instance comprises DNA or RNA. Methods for the preparation and administration of a compound or pharmaceutical composition are known in the art, as well as suitable carriers for pharmaceutical compositions. Likewise, methods for gene therapy are known by the person skilled in the art. In the art, many vectors and protocols are provided allowing the person skilled in the art to perform an optimal therapy for each application.

A use of at least a functional part of a monocarboxylate transporter protein, or of a nucleic acid encoding at least a functional part of a monocarboxylate transporter protein, in a method for treating a disorder such as a thyroid hormone related disorder such as a disorder of thyroid hormone metabolism, non-thyroidal illness, obesity or cardiovascular illness is also herewith provided. In one embodiment, the use comprises gene therapy.

In yet another aspect, the invention provides a method for altering transport of a thyroid hormone or a functional part, derivative and/or analogue thereof across a membrane comprising providing the membrane with a monocarboxylate transporter protein or a functional part, derivative and/or analogue thereof, and/or with a molecule capable of specifically binding a monocarboxylate transporter protein or a functional part, derivative and/or analogue thereof, or a ligand of the monocarboxylate transporter protein, and/or with a molecule capable of specifically binding a nucleic acid sequence encoding a monocarboxylate transporter protein or a functional part, derivative and/or analogue thereof, or encoding a ligand of the monocarboxylate transporter protein or functional part, derivative and/or analogue. As has been outlined before, an MCT protein and a molecule capable of specifically binding at least part of an MCT protein or at least part of a ligand of an MCT protein, or a nucleic acid encoding the MCT and/or ligand, are particularly suitable for altering thyroid hormone transport across a membrane, preferably across a plasma membrane. In a preferred embodiment the MCT protein comprises MCT-S.

An MCT protein or a nucleic acid encoding at least a functional part of an MCT protein, preferably an MCT-8 protein, can be used in a method for detecting a pharmaceutical compound. The identification of MCT protein involved in the uptake of thyroid hormone is not only important in the study of the regulation of these processes in health and disease, but it also provides for the detection and/or development of thyroid hormone agonists and antagonists which are beneficial in the treatment of conditions such as obesity and cardiovascular diseases. Ligands or blockers of MCT proteins are important candidate drugs for the development of pharmaceutical compositions acting as a (tissue specific) agonist or antagonist of thyroid hormone activity, which are herewith provided. Methods to identify and/or generate a ligand or blocker of a protein, such as an MCT protein, are known in the art. One can for instance use an assay allowing thyroid hormone and a candidate drug compound to compete for transport across a membrane by an MCT protein. One can also provide a host cell with a nucleic acid sequence encoding at least part of the protein and allow for expression of the nucleic acid sequence. Transport of thyroid hormone in the presence of a candidate drug compound across the membrane of the host cell can subsequently be determined. The invention therefore provides a use of at least a functional part of a monocarboxylate transporter protein, or a nucleic acid encoding at least a functional part of a monocarboxylate transporter protein, in a method for detecting and/or generating a pharmaceutical compound. A preferred embodiment provides a use of the invention wherein the monocarboxylate transporter protein comprises monocarboxylate transporter protein-S.

In one embodiment, such ligands or blockers comprise a peptide derived from a nucleic acid or fragment thereof encoding an MCT protein. To derive peptides which act as ligand (agonist) or blocker (antagonist) is a skill known in the art, for example, one can use Pepscan techniques or replacement mapping techniques as well as for example phage-display techniques and screening of combinatorial libraries, allowing identification of active sites in a polypeptide sequence. In particular, the invention provides a peptide at least comprising an active site capable of binding to or influencing or interfering with the binding and/or transporting of a ligand (preferably of a thyroid hormone nature or functionally equivalent thereto) of a thyroid hormone binding site or part thereof, of an MCT protein, preferably MCT-8.

Furthermore, the invention provides a (synthetic) antibody or other binding molecule specifically directed against an MCT protein, preferably MCT-8, or at least binding to or interfering with the binding of a ligand of a thyroid hormone binding site or parts thereof, of the MCT protein. Generating antibodies or other binding molecules is a skill available in the art, and can be done with classical immunological techniques as well as for example with phage-display techniques.

The invention also provides a bioassay or method to identify the candidate drug agonists or antagonists, for example for use in a pharmaceutical composition for treating obesity, heart failure or (tissue specific) hypo- or hyper-thyroidism. Candidate drugs, often first selected or generated via combinatorial chemistry or comprising a peptide as provided by the invention, can now be tested and identified using a method provided by the invention. Such a candidate drug or compound can for example be tested on and selected for its effect on T3 uptake by an MCT protein. For use in brain cells, where T3 is autonomously produced, a candidate drug or compound can for example be tested on and selected for its effect on T4 uptake as a precursor for T3. As for example can be seen in several of the figures in the description, the invention provides methods and means to measure thyroid hormone cellular uptake by an MCT protein, and regulation thereof.

The invention thus provides in one aspect a bioassay to identify or detect a candidate drug capable of binding to or influencing a plasma membrane polypeptide capable of transporting a thyroid hormone, wherein the polypeptide comprises a monocarboxylate transporter protein or a functional part, derivative and/or analogue thereof.

In a preferred embodiment, the invention provides a bioassay of the invention using at least a functional part of a monocarboxylate transporter protein, a nucleic acid encoding at least a functional part of a monocarboxylate transporter protein, a molecule capable of specifically binding at least part of the monocarboxylate transporter protein or a ligand thereof, a cell comprising the nucleic acid and/or a cell comprising at least part of the monocarboxylate transporter protein.

In a most preferred embodiment, a bioassay of the invention is provided wherein the monocarboxylate transporter protein comprises monocarboxylate transporter protein-S.

The invention also provides an isolated or recombinant monocarboxylate transporters protein or a functional part, derivative and/or analogue thereof. Such functional part for instance comprises a thyroid hormone binding site or at least a part of such a site, which often comprises one or more peptides within the large polypeptide. In one embodiment the protein comprises rat monocarboxylate transporter-S protein. Expressing recombinant protein and isolating and purifying a protein or fragment thereof is a skill available in the art. For instance, a vector can be provided with a nucleic acid sequence encoding a monocarboxylate transporter-S protein or a functional part, derivative and/or analogue thereof. The vector can be administered to a host cell, for instance by a gene delivery vehicle. If the cell is capable of expressing the MCT protein, the MCT protein can be produced and isolated by methods known in the art.

A vector comprising a nucleic acid encoding a monocarboxylate transporter protein, preferably MCT-8, or a functional part, derivative and/or analogue thereof is also suitable for gene therapy. The same is true for a vector comprising a nucleic acid sequence encoding a molecule capable of specifically binding a monocarboxylate transporter protein, preferably MCT-8, or a functional part, derivative and/or analogue thereof, or for a vector capable of specifically binding a ligand of the monocarboxylate transporter protein. Such vectors are therefore also herewith provided.

The invention also provides a gene delivery vehicle comprising a vector of the invention. Methods for generating a gene delivery vehicle comprising a certain nucleic acid of interest are known in the art. A gene delivery vehicle of the invention is very suitable for treatment of a disorder such as a disorder of thyroid metabolism, non-thyroidal illness, obesity or cardiovascular illness with gene therapy.

Now that the invention provides the knowledge that monocarboxylate transporter protein is involved in thyroid hormone transport, it has become possible to investigate whether an individual suffers from, or is at risk of suffering from, disorders of thyroid hormone metabolism such as for instance brain disorders, tissue metabolism disorders, non-thyroidal illness, obesity and/or cardiovascular illness. It can be estimated whether thyroid hormone transport is adversely affected. This can for instance be done by determining whether an individual comprises a mutation in a nucleic acid of its genome encoding a monocarboxylate transporter protein or a functional part, derivative and/or analogue thereof, or a ligand of the monocarboxylate transporter protein. A mutation in the nucleic acid, such as for instance a substitution, deletion or addition of at least one nucleotide, is indicative for impaired expression of the monocarboxylate transporter protein or a functional part, derivative and/or analogue thereof, or a ligand of the monocarboxylate transporter protein, resulting in impaired thyroid hormone transport.

This is confirmed by an investigation of a patient suffering from a thyroid hormone disorder characterized by high serum T3 and TSH titers, low T4 and rT3 titers, and mental disorder. Analysis of the MCT-S gene of the patient has revealed that the gene lacks exon 1, while exons 2-6 are present. This shows that a mutation of a monocarboxylate transporter protein gene is involved with a thyroid hormone disorder. Analysis of the monocarboxylate transporter protein gene, or a gene of a ligand thereof, therefore provides an important diagnostic tool for detecting thyroid hormone disorders. Methods for detecting a mutation in a nucleic acid are known in the art. For instance, total nucleic acid of a sample may be isolated using a known method in the art and the nucleic acid may subsequently be amplified, preferably using specific primers for a gene encoding at least one monocarboxylate transporter protein or a ligand thereof. Subsequently, mutations in the gene can be detected, for instance using specific probes, by sequencing amplified product, etcetera.

The invention therefore provides a method for determining whether an individual is suffering from, or is at risk of suffering from, a thyroid hormone related disorder, non-thyroidal illness, obesity and/or cardiovascular illness, comprising determining whether the individual comprises a mutation in a nucleic acid sequence encoding a monocarboxylate transporter protein or a functional part, derivative and/or analogue thereof, and/or encoding a ligand of the monocarboxylate transporter protein or functional part, derivative and/or analogue. In a preferred embodiment the mutation comprises a deletion. In a more preferred embodiment the mutation comprises a deletion of at least one exon.

In a further preferred embodiment a method of the invention is provided wherein the monocarboxylate transporter protein comprises monocarboxylate transporter protein-S.

A diagnostic kit comprising suitable means for detecting a mutation in a nucleic acid sequence encoding a monocarboxylate transporter protein or a functional part, derivative and/or analogue thereof, and/or encoding a ligand of the monocarboxylate transporter protein or functional part, derivative and/or analogue is also herewith provided.

### BRIEF DESCRIPTION OF THE DRAWING

FIG. 1: Transport of iodothyronines by MCTS in oocytes

FIG. 2: Transport of amino acids by MCT8 in oocytes

FIG. 3: Saturation of T4 transport by MCT8 in oocytes

FIG. 4: Time course of T3 uptake by MCT8 in oocytes

FIG. 5: Amino sequence alignment of rat, mouse and human MCT8. Alignment was performed using the ClustalW program (DNASTAR LaserGene software). Identity between 3 species are indicated in black, and identity between 2 species in gray.

FIG. 6: Western blot to show expression of FLAG-tagged MCT8 in X. laevis oocytes. Plasma membrane preparations were prepared from oocytes 3 days after injection with water (lane 1), MCT1 (lane 2) or MCT8-FLAG cRNA (lane 3 and 4) and subjected to SDS-PAGE and Western blotting with the anti-FLAG antibody or the anti-MCT1 antibody as described in the experimental section.

FIG. 7: Detection of FLAG-tagged MCT8 protein at the cell surface of the oocytes, Oocytes were fixed, sectioned and prepared for immunofluorescence microscopy (anti-FLAG antibody) 3 days after injection with MCT8-FLAG cRNA or water (control) as described in the experimental section.

FIG. 8: Time course of T₃ and T4 uptake in oocytes. Oocytes were injected with MCT8 cRNA and after 3 days incubated for 2-60 mm at 25 C with 10 nM [1ZSI]T3 (A) or [125]jT4 (B) in medium with or without Nat In the latter NaCl was replaced by choline chloride.

FIG. 9: MCT8 cRNA concentration-dependent uptake of T₃ and T4. Oocytes were injected with 0-11.5 ng MCT8 cRNA and after 3 days incubated for 2 mm at 25 C with 10 nM t3 (.) or Ti (a).

FIG. 10: Temperature-dependent uptake of T₃ and T4 by MCT8. Oocytes were incubated for 2 mm at 4, 15, 25 or 37 C with 10 nM T₃ (●) or T4 (∘). Net uptake of T3 or T4 in MCT8 cRNA-injected oocytes is corrected for the corresponding uptake in uninjected oocytes.

FIG. 11: Albumin-dependent uptake of T3. Oocytes were incubated for 2 mm at 25 C in standard uptake solution with 10 nM t3 without albumin, or supplemented with 0.1 % or 0.5 % albumin (a). Uninjected oocytes were used as controls (∘). Numbers indicate fold stimulation by MCT8.

FIG. 12: Ligand-dependent transport by MCT8. MCT8 cRNA injected oocytes were incubated for 2-60 mm at 25 C with 10 nM [’25I]iodothyronines or 10 pM [3Hjamino acids (Tyr, Trp, Leu and Phe) (a). Uninjected oocytes were used as controls (a). The uptake of the different putative ligands is expressed per mm.

FIG. 13: Uptake and metabolism of rT₃. Oocytes were incubated for 2-60 mm at 25 C with 10 nM rT₃. At each time point, uninjected oocytes (left panel) and MCT8 cRNA-injected oocytes (right panel) were homogenized, and homogenates and medium samples were analyzed as is described in experimental procedures.

FIG. 14: Kinetics of MCT8 mediated rT₃, T3 and T4 uptake. Oocytes were incubated with increasing rT₃, T₃ or T4 concentrations (1 nM-30 pM). Net transport by MCT8 was calculated by subtracting uptake in uninjected oocytes from that in MCT8 cRNA-injected oocytes. Uptake is expressed as fmol/oocyte*mm. Kinetics were performed using the Slide Write Plus Program (Advanced Graphics Software).

### DETAILED DESCRIPTION

The invention is further explained in the following examples. The examples do not limit the scope of the invention; they merely serve to exemplify the invention.

### EXAMPLES

### Example 1

Transport of iodothyronines by MCT8 in oocytes

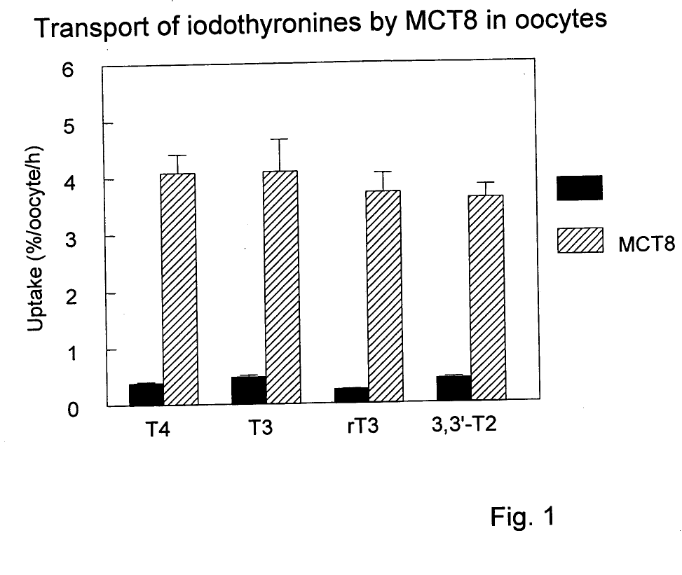

Xenopus oocytes were isolated and injected with 4.6 ng rat MCTS cRNA. After 3 days, groups of 10 injected or uninjected oocytes were incubated for 60 mm at 25 C with 10 nM radioactive iodothyronines in 0.1 ml medium. Uptake of iodothyronines was determined as previously described (18). The results are shown in FIG. 1.

### Example 2

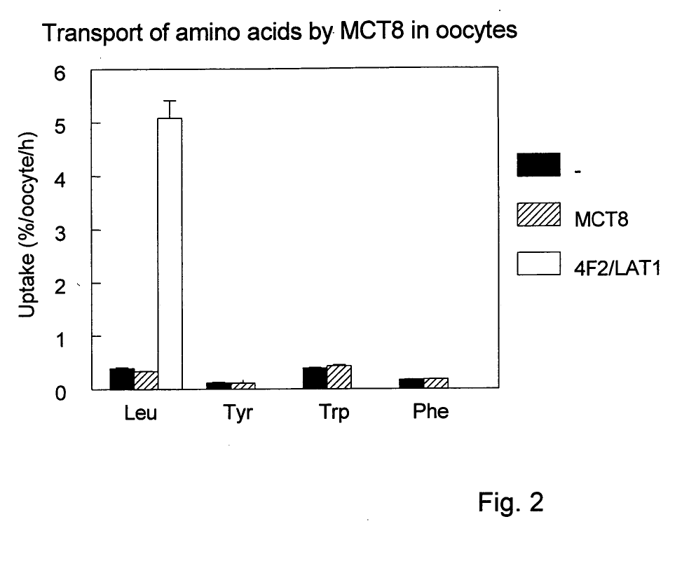

Transport of amino acids by MCTS in oocytes Xenopus oocytes were isolated and injected with cRNA coding for rat MCT8 or with cRNAs coding for the heavy chain (4F2) and the light chain (LAT1) of the heterodimeric human L-type amino acid transporter. After 3 days, groups of 10 injected or uninjected oocytes were incubated for 60 mm at 25 C with 10 μM radioactive Leu, Tyr, Trp or Phe in 0.1 ml medium. Amino acid uptake was determined as previously described (18). The results are shown in FIG. 2.

### Example 3

Saturation of T4 transport by MCTS in oocytes

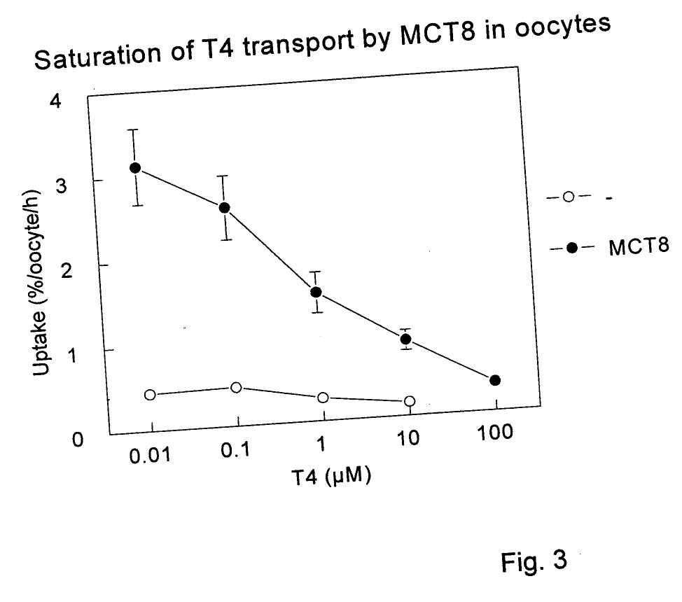

Xenopus oocytes were isolated and injected with rat MCTS cRNA. After 3 days, groups of 10 injected or uninjected oocytes were incubated for 60 mm at 25 C with radioactive T4 and increasing concentrations of non-radioactive T4 in 0.1 ml medium. T4 uptake was determined as previously described (18). The results are shown in FIG. 3.

### Example 4

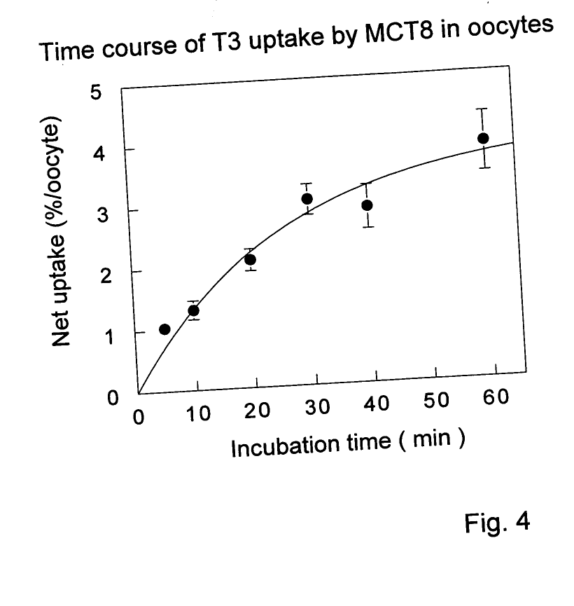

Time course of T3 uptake by MCTS in oocytes Xenopus oocytes were isolated and injected with rat MCTS cRNA. After 3 days, groups of 10 injected or uninjected oocytes were incubated for 5-60 mm at 25 C with 10 nM radioactive T3 in 0.1 ml medium. T3 uptake was determined as previously described (18). The results are shown in FIG. 4.

Procedures

Materials—Nonradioactive L-iodothyronines, 3,3′,5-triiodothyroacetic acid (Triac) and N-bromoacetyl-3,3′,5-triiodothyronine (BrAcT5) were obtained from Henning Berlin. D-iodothyronines, Phe and Tyr were purchased from Sigma, Leu was obtained from Merck, and Trp and bromosulfophthalein (BSP) were purchased from Fluka. [3′,5′˜125I]T4, [3′.125I]Ts and carrier-free Na′251 were purchased from Amersham Biosciences. All other 125I-labeled compounds were prepared as previously described (21). 3H-labeled Leu, Phe, Tyr, and Trp were purchased from Amersham Biosciences. All other chemicals were of reagent grade.

Cloning of rat MCT8-Primers for RT-PCR were designed to regions of high homology in the 5′ and 3′ untranslated region (UTR) sequences of human (05315) and mouse (AF045692) MCTS. The sense primer was 5′ AGCTCTCGAGCGGCAAGCCACAGTCAG 3′ (SEQ ID NO: __) corresponding to the mouse sequence from nucleotide (nt) 145 (the coding sequence starts at nt 175) and contained a XhoI restriction site (underlined). The antisense primer was 5′ AAATGCGGCCGCTTCTCCGTTGGGGTCT 3′ (SEQ ID NO: __) corresponding to the mouse sequence ending at nt 2242 (the coding sequence ends at nt 1872) and contained a NotI restriction site (underlined). Isolated rat liver mRNA was in vitro reverse transcribed and amplified using the reverse transcription system from Promega and subjected to PCR using the above primers. The 2038 bp product was ligated into pGEMT-Easy and sequenced.

Insertion of a FLAG-tagged MCT8 construct into the oocyte expression vector pGEM-HeJuel- For expression in oocytes it was decided to append an 8 amino acid “FLAG” epitope to the C-terminus of MCT8 in order to allow detection of expression by immunofluorescence microscopy and Western blotting. For this purpose the stop codon of MCT8 was removed by performing PCR using the same sense primer as above, but with the modified antisense primer, 5′ ACAGCGGCCGCAAATGGGCTCTTCAGGTGTTG 3′ (SEQ ID NO:__) which lacks the stop codon. This PCR product was ligated into pGEMT-Easy before being excised with EcoRI and ligated into the FLAG vector, pCMV-Tag4A (Stratagene), which had previously been digested with the same restriction enzyme and dephosphorylated. The FLAG epitope-tagged MCT8 was then ligated into the Xenopus oocyte expression vector pGEM-Hejuel which contains the 5′- and 3′-UTRs of the Xenopus B-globin flanking a multiple cloning site. The stability of the transcribed MCT8 mRNA sandwiched between the untranslated globin sequence is likely to be enhanced in the oocyte as was found to be the case for other MCTs (22). The MCT8-FLAG insert was prepared using PCR with primers flanking the MCTS-FLAG insert in pCM˜V˜Tag4A and containing suitable restriction sites for the insertion into the pGEM-HeJuel vector. The sense primer was 5′ GCGGGGATCCACACGTCAGTCCCCTAGCCA 3′ (SEQ ID NO:__) and contained a BamHI restriction site (underlined) whilst the antisense primer was 5′ CVI′ATCTAGATAAGGTACCGGGCCCTACT 3′ (SEQ ID NO:__) and contained an XbaI restriction site (underlined). Following successful PCR amplification, the product was digested with BamHI and XbaI and ligated into the pGEM-HeJuel vector digested with the same restriction enzymes. The correct identity of the product was established using an EcoRI digest and confirmed with sequencing.

X. laevis oocyte expression—MCT8 cRNA was obtained by in vitro transcription using the AMPLISCRIBE HIGH YIELD™ T7 RNA transcription kit (Epicentre) after linearization of pGEM-HeJuel containing the MCTS cDNA with NotI. Oocytes were isolated and allowed to recover overnight at 18 C in modified Barth's solution containing 20 IU/ml penicillin and 20 gg/ml streptomycin as described before (23). The next day, oocytes were injected with cRNA coding for MCT8, and further incubated for 3 days at 18 C in modified Barth's solution until analysis. Uninjected or water injected oocytes were used as controls.

Western blotting of oocyte membranes—Crude oocyte membranes were prepared using 10 oocytes harvested 3 days after microinjection with MCTS or MCT1 cRNA or the equivalent volume of water. Oocytes were suspended in 500 pl of buffer (10 mM HEPES, 83 mM NaCl, 1 mM MgCl2, pH 7.9, containing 4 mg/ml pepstatin A, leupeptin, antipain and 0.5 mM phenylmethylsulfonyl fluoride and benzamidine) and homogenized by vigorous vortex mixing followed by several passes through an 18 gauge needle. The homogenate was centrifuged at 2000 rpm for 5 mm at 4 C and the supernatant was collected, whilst the pellet was re-homogenized in 500 pl buffer and centrifuged as before. Both the supernatants were then mixed and centrifuged at 100,000 g for 60 mm at 4 C and the pellet resuspended in 30 pl buffer and 30 pl of SDS-PAGE sample buffer. Following separation by SDS-PAGE Western blotting was performed using mouse anti-FLAG monoclonal antibody (Sigma) or the specific MCT1 antibody with detection by enhanced chemiluminescence (ECL) as described previously (24).

Preparation and staining of oocytes for immunofluorescence confocal microscopy—Oocytes were first embedded in pieces of chicken liver and then placed on pieces of cork, covered in O.C.T. embedding compound (Tissue-Tek, Sakura Finetek Europe BV, The Netherlands) and frozen in liquid-nitrogen cooled isopentane. Frozen sections (5 μm) were cut, placed on silanized slides and air-dried at room temperature for 1 h before fixing with ice-cold acetone for 10 mm. Permeabilization and staining were then carried out as previously described (24) using a mouse monoclonal anti-FLAG antibody and TRITC-conjugated anti-mouse IgG secondary antibody. Samples were mounted with Mowiol (Calbiochem) and examined with a Leica TCS-NT confocal scanning microscope (63×1.32 NA oil immersion objective).

Transport measurements in oocytes—Oocytes were washed and preincubated at 18 C in standard uptake solution (100 mM NaCl, 2 mM KCl, 1 mM CaCl2, 1 mM MgCl2, 10 mM HEPES, and 10 mM Tris, pH 7.5). Usually, transport was tested by incubation of groups of 5-10 oocytes for 2-60 mm at 25 C with 10 nM [1251]iodothyronines or 10 μM [3H]amino acids in 0.1 ml standard uptake solution. The possible Na⁺ dependence of transport was tested by preincubation and incubation in Na⁺-free uptake solution in which NaCl was replaced by choline chloride. The influence of the temperature on the uptake rate was tested by the incubation of oocytes with uptake solutions at different temperatures between 4 and 37 C. The influence of albumin on uptake of T3 was tested by the addition of 0.1% and 0.5% BSA to the standard uptake solution. The incubation was stopped by aspiration of the medium, and oocytes were washed 4 times at 4 C with standard uptake solution containing 0.1% BSA.

The substrate specificity of MCT8 was investigated by incubation of oocytes with different putative radioactive ligands, including T4, T3, 3,3,5′-triiodothyronine (rT₃), 3,3′.diiodothyronine (T2), Na-sulfonated T4 (T4 sulfamate, T4NS) and 4′-OH-sulfonated T4 (T4 sulfate, T4S), and the amino acids Leu, Phe, Tyr and Trp. Specificity of transport of 1251-T4 and 12˜I.T3 was measured in the presence of putative competitors, including 10 μM unlabeled iodothyronine derivatives such as D- and L-iodothyronines, Triac and BrAcT3, and 100 pM Tyr, Trp or BSP.

Analysis of rT3 metabolism in oocytes—Groups of 10 oocytes were incubated for 2-60 mm at 25 C with 10 nM ['25IIrTa. After incubation, medium was collected, and 2 groups of 5 oocytes were counted separately and homogenized in 0.1 ml 0.1 M NaOH as described before (25). Lysates were cleared by centrifugation. Lysates (in duplicate) and incubation media were acidified with 0.1 M HCl and analyzed by Sephadex LH-20 chromatography (21). The different products were successively eluted with 0.1 M HCl (iodide), water (conjugates), and 1% NH4OH in ethanol (iodothyronines).

Transport kinetics—Saturation of iodothyronine uptake in MCT8 cRNA injected oocytes was analyzed in incubations containing labeled and unlabeled T4, T3, or rT3 at final concentrations of 1 nM-30 μM. Apparent Km values were calculated by fitting the plot of uptake rate (v) versus ligand concentration (5) to the Michaelis Menten equation: v=Vmax/(1+Km/S), wherein Vmax is the maximum uptake rate, and Km the Michaelis constant. Calculations were performed using the Slide Write Plus program version 5.01 (Advanced Graphics Software).

Statistics. Results are expressed as means±SEM. Statistical significance was determined using the Student's t test for unpaired observations.

Results

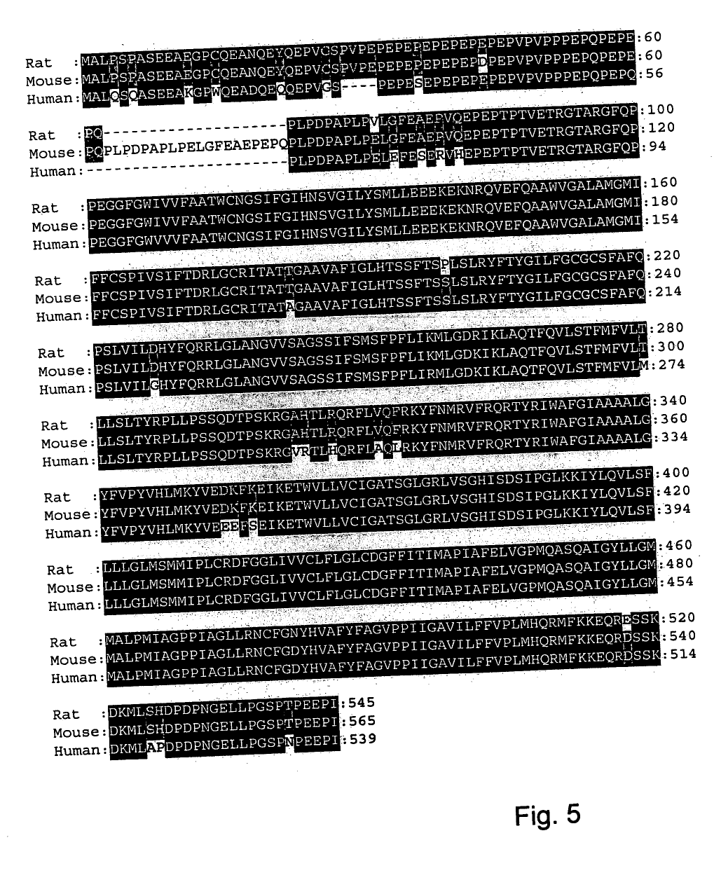

Cloning of rat MCT8—The coding sequence of rat MCT8 was cloned using RT-PCR from rat liver mRNA as described in the experimental section and has been assigned the accession code NIVIj47216 (gi:22219453). The translated protein sequence is shown in FIG. 5 where it is aligned with human (1105315) and mouse (AF045692) MCT8 sequences. As would be predicted, the rat and mouse sequences show very few differences with only four amino acids changes and the insertion of a 20 amino acid repeat in the mouse PEST sequence which is absent in the rat and human sequences. The predicted molecular mass of the protein is 60.1 kDa.

Expression of the FLAG-tagged MCT8 in Xenopus oocytes—We initially sought to express the MCT8 in oocytes but the antibody we raised to the C-terminus of the protein (which we have used successfully for production of antibodies against other MCTs) failed to detect native and recombinant rat MCT8. Thus, we expressed MCT8 with a FLAG epitope attached to the C-terminus as described in the experimental section. The cloning strategy used means that the C-terminus is extended by the following sequence (FLAG-tag underlined) CAAVITSEFDIKLIDTVDLEDYKDIJDDK (SEQ ID NO:__) giving a predicted molecular mass 63.3 kDa.

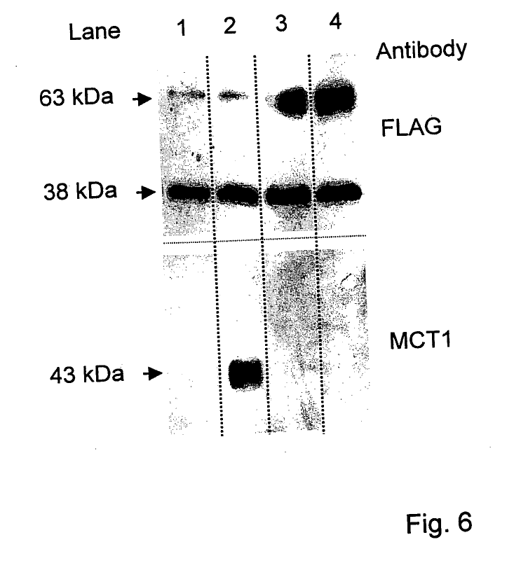

In FIG. 6, we present Western blots using an anti-FLAG antibody of membranes derived from oocytes injected with water (control), MCT1 (26) or MCT8-FLAG cRNA. A band of about 63 kDa is present only in the membranes from the MCT8 cRNA-injected oocytes (lanes 3 and 4) confirming the presence of the expressed FLAG-tagged MCT8. The band at 38 kDa is a FLAG-sensitive band in X. laevis oocytes. Plasma membranes from oocytes injected with MCT1 cRNA showed no response to the FLAG antibody but did show a 43 kDa protein corresponding to MCT1 with the specific MCT1 antibody (lane 2, lower part). The latter failed to detect the MCT8 as would be expected in view of the lack of similarity between the C-termini of MCT8 and MCT1.

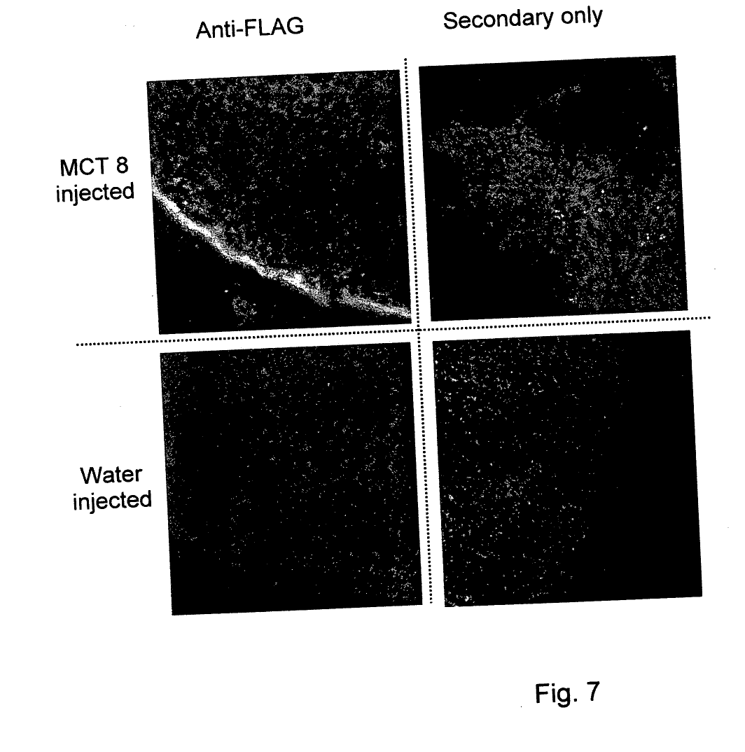

In order to confirm that MCT8 was expressed at the plasma membrane we performed immunofluorescence microscopy on sections of oocytes, again using the anti-FLAG antibody. The data are shown in FIG. 7 and reveal that MCT8 is strongly expressed at the plasma membrane. Water-injected oocytes showed no such expression; nor did the secondary antibody alone detect any protein at the plasma membrane.

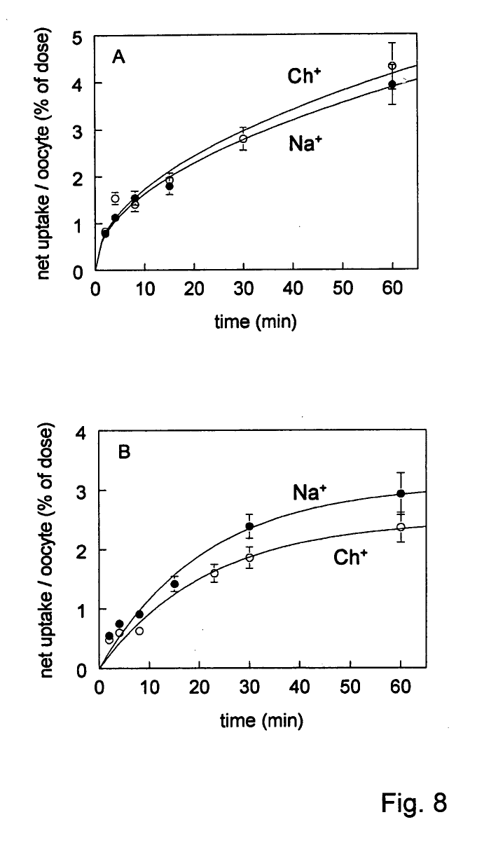

Functional characterization—Transport studies were performed to investigate the function of MCT8 expressed in X. laevis oocytes. FIG. 8 shows the time course of uptake of T3 and T.1 in uninjected oocytes and in oocytes injected with 4.6 ng MCT8 cRNA. Expression of MCT8 induced a ˜10-fold increase in initial uptake of T3 and T4 compared with uninjected oocytes. This graph also shows that the uptake of T3 and T4 into MCT8 cRNA-injected oocytes was only linear for the first 4 mm. Therefore, all further transport experiments were performed at 2 mm incubations. The difference in uptake of T3 and T4 in oocytes injected with MCT8 cRNA is not statistically significant. Transport of t3 was independent of Na⁺ as the same results were obtained using medium with choline chloride instead of NaCl (FIG. 8A). However, T4 transport by MCT8 showed a modest but consistent inhibition in the absence of Na⁺ (FIG. 8B).

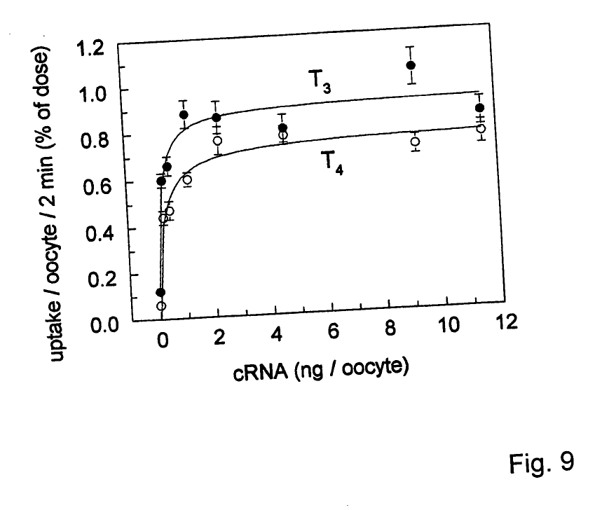

FIG. 9 shows the influence of the amount of cRNA injected on the uptake of 10 nM T3 and 1′4. The results indicate that the lowest amount of MCT8 cRNA (0.23 ng) injected already induced a 5-fold increase in T3 transport and a 7.3-fold increase in T4 transport. Maximum induction of T3 and T4 transport was found after injection of 1.15-2.3 ng of MCT8 cRNA. Therefore, oocytes were further injected with 4.6 ng MCT8 cRNA for maximum induction of iodothyronine transport.

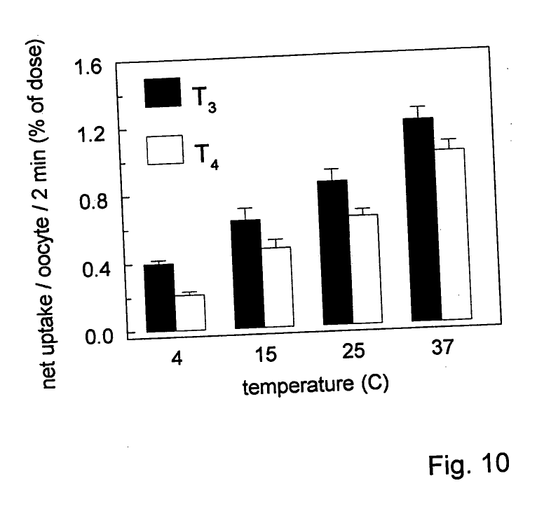

To test the temperature dependence of MCT8-mediated iodothyronine transport, oocytes were incubated with 10 nM [1251]T₃ or [1251]T4 for 2 mm at 4-37 C. FIG. 10 shows significant uptake for T3 and T4 into the oocytes at 4 C, with marked increases if the temperature was increased to 15, 25 and 37 C, which is above the usual ambient temperature for frog oocytes. Exposure to 37 C was tolerated by the oocytes during this short incubation time; prolonged incubation at 37 C resulted in disintegration of the cells. The results show identical temperature dependence of transport of T4 and T3 by MCT8.

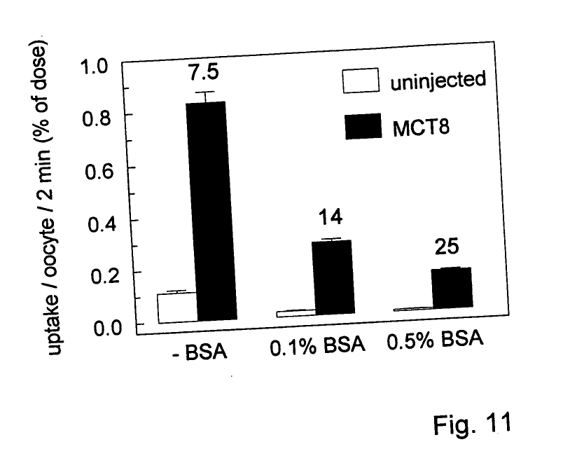

FIG. 11 shows the effects of addition of 0.1% and 0.5% BSA on the uptake of T₃ in oocytes. Both in uninjected oocytes and in MCT8 cRNA-injected oocytes, T₃ uptake was decreased concentration-dependently by BSA. However, the fold stimulation of T₃ uptake by MCT8 increased from 75 in the absence of BSA to 14 in the presence of 0.1% BSA, and further to 25 in the presence of 0.5% BSA.

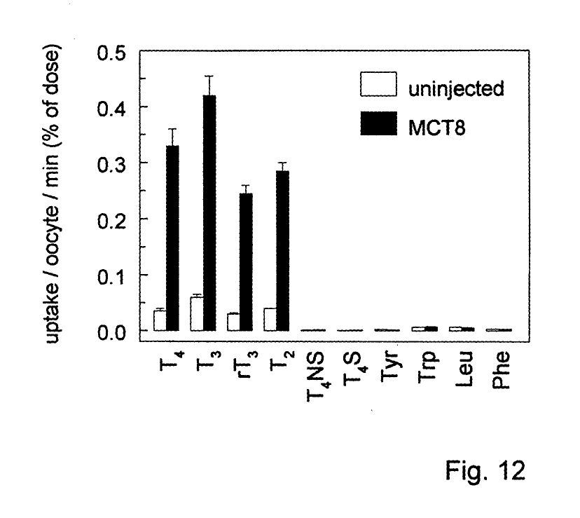

Substrate specificity—The substrate specificity of MCT8 was investigated by incubation of oocytes with different putative radioactive ligands, including T4, T3, rT₃, T2, TiNS and T4S, and the amino acids Leu, Phe, Tyr and Trp. FIG. 12 shows that in contrast to the rapid uptake of the different iodothyronines, T4NS and T4S and the amino acids are not transported by MCT8. MCT8 failed to transport the different amino acids at concentrations (1-100 pM) showing facile transport by the LAT1 (18) and TAT1 (19, 20) amino acid transporters. We have also been unable to demonstrate any transport of ['4C]lactate, whereas in oocytes injected with rat MCT1 cRNA, transport of [14C]lactate was greatly stimulated compared to control oocytes, but no additional uptake of [125I1T4 was observed (data not shown).

The specificity of iodothyronine transport by MCTS was further addressed by investigating the uptake of [125I]T₃ and ['25I]T4 in the absence or presence of structurally related compounds (Table 1). Uptake of labeled T4 and T3 by MCT8 was potently inhibited by 10 pM unlabeled L-T4, D-T4, L-T3 and DT3, indicating that the interaction of T4 and t3 with MCT8 is not stereospecific. lodothyronine uptake by MCT8 is also potently inhibited by t3 analogs where the aNH2 group is blocked (BrAcT3) or deleted (Triac), indicating that this aNH2 group is not important for interaction of iodothyronines with MCT8. The T-type amino acids Tyr and Trp hardly affected iodothyronine uptake by MCT8. The organic anion BSP proved to be a potent inhibitor. This, in contrast, to the weak effects of the bile acid taurocholate and the organic anion transport inhibitor probenecid (data not shown). In general, T3 transport by MCT8 shows less inhibition by these compounds than MCT8-mediated T4 transport. It is remarkable that ligands and potent inhibitors of MCT8 all carry halogen atoms.

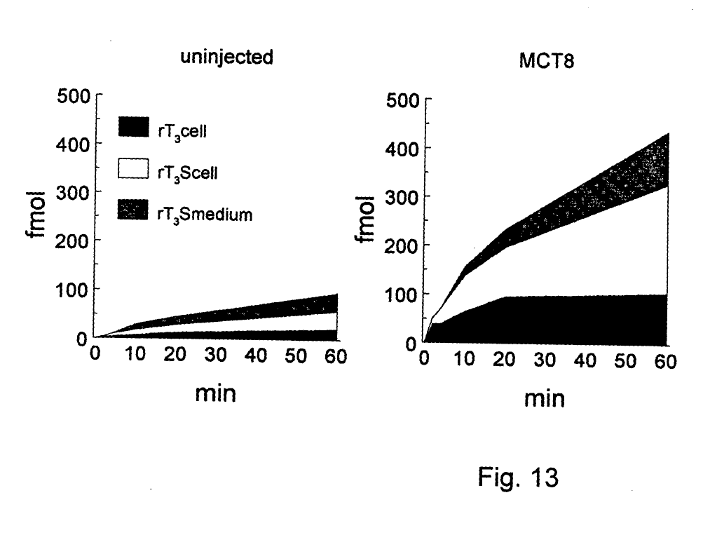

Metabolism of rT₃—Previous studies have shown that X. laevis oocytes actively metabolize rT₃ by sulfation. Since sulfotransferases are located intracellularly, we have used this property to investigate rT₃ internalization by oocytes. FIG. 13 shows the time course of rT₃ uptake and subsequent metabolism in native and MCT8-expressing oocytes. At several time points, uninjected and MCT8 cRNA-injected oocytes are homogenized, and the homogenates and medium samples are analyzed for rT₃ and rT₃ sulfate (rT₃S) content. It is shown that already after 2 mm of incubation, rT₃S is formed intracellularly in the MCT8 cRNA-injected oocytes. After 10 mm of incubation, release of rT₃S into the medium is observed. The production rate of rT₃S is much higher in MCT8 cRNA-injected oocytes than in uninjected cells. These findings demonstrate that MCT8-mediated uptake of iodothyronines indeed represents the internalization of these compounds.

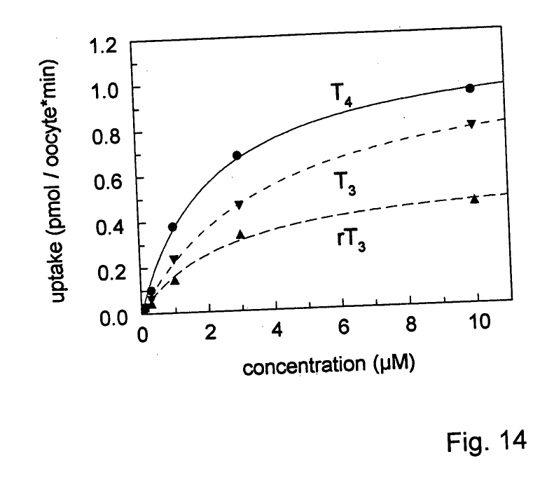

Transport kinetics—The kinetics of MCT8-mediated iodothyronine transport were analyzed by incubation of oocytes with increasing T4, t3 or rt3 concentrations (1 nM-30 pM). Net transport by MCT8 was calculated by subtracting uptake in uninjected oocytes from that in MCT8 cRNA-injected oocytes. The results showed that transport of T4, T3 and rT₃ by MCT8 was saturable. Michaelis-Menten analysis of the data provided apparent Km values of 4.7 pJv1 for T4, 4.0 pM for t3 and 2.2 μM for rt3 (FIG. 14).

### References

1. Larsen, P. R. and M. J. Berry. 1995. Nutritional and hormonal regulation of thyroid hormone deiodinases. Annu. Rev. Nutr. 15: 323-352.
2. Leonard, J. L. and J. Kohrle. 1996. Intracellular pathways of iodothyronine metabolism. In The Thyroid, L. E. Braverman and R. Utiger, editors. Lippincott-Raven, Philadelphia, 125-161.
3. St. Germain, D. L. and V. A. Galton. 1997. The deiodinase family of selenoproteins. Thyroid 7: 655-688.
4. Hennemann, G. and T. J. Visser. 1997. Thyroid hormone synthesis, plasma membrane transport and metabolism. In Handbook of Experimental Pharmacology, Vol 128; Pharmacotherapeutics of the Thyroid Gland, A. P. Weetman and A. Grossman, editors. Springer, Berlin, 75-117.
5. Oppenheimer, J. H., H. L. Schwartz and K-A. Strait. 1996. The molecular basis of thyroid hormone actions. In The Thyroid, L. E. Braverman and R. Utiger, editors. Lippincott-Raven, Philadelphia, 162-184.
6. Hennemann, G., M. E. Everts, M. de Jong, C. F. Lim, E. P. Krenning and R. Docter. 1998. The significance of plasma membrane transport in the bioavailability of thyroid hormone. Clin. Endocrinol. 48:1-8.
7. Hagenbuch, B., B. Stieger, M. Foguet, H. Lübbert and P. J. Meier. 1991. Functional expression cloning and characterization of the hepatocyte Na+/bile acid cotransport system. Proc. Natl. Acad. Sci. USA. 88: 10629-10633.
8. Jacquemin, E., B. Hagenbuch, B. Stieger, A. W. Wolkoff and P. J. Meier. 1994. Expression cloning of a rat liver Na+-independent organic anion transporter. Proc. Nati. Acad. Sci. USA 91: 133-137.
9. Hagenbuch, B. 1997. Molecular properties of hepatic uptake systems for bile acids and organic anions. J. Membrane Biol. 160:1-8.
10. Meier, P. J. 1995. Molecular mechanisms of hepatic bile salt transport from sinusoidal blood into bile. Am. J. Physiol. 269: G801-G812.
11. Schroeder, A., U. Eckhardt, B. Stieger, R. Tynes, C. D. Schteingart, A. F. Hofmann, P. J. Meier and B. Hagenbuch. 1998. Substrate specificity of rat liver Na˜-bile salt cotransporter in Xenopus laevis oocytes and CHO cells. Am. J. Physiol. 274: G370-G375.
12. Bossuyt, X., M. Muller and P. J. Meier. 1996. Multispecific amphipathic substrate transport by an organic anion transporter of human liver. J. Hepatol. 25:733-738.
13. Bossuyt, X., M. Muller, B. Hagenbuch and P. J. Meier. 1996. Polyspecific drug and steroid clearance by an organic anion transporter of mammalian liver. J. Pharmacol. Exp. Ther. 276:891-896.
14. Kanam, N., R. Lu, Y. Bao, A. W. Wolkoff and V. L. Schuster. 1996. Transient expression of oatp anion transporter in mammalian cells: identification of candidate substrates. Am. J. Physiol. 270: F319-F325.
15. Kullak-Ublick, G. A., T. Fisch, M. Oswald, B. Hagenbuch, P. J. Meier, U. Beuers and G. Paumgartner. 1998. Dehydroepiandrosterone sulfate (DHEAS): identification of a carrier protein in human liver and brain. FEBS Lett. 424:173-176.
16. Henneman, G; Docter, R; Friesema, E; De Jong, M; Krenning, E; and Visser, Th. 2001. Plasma membrane transport of thyroid hormones and its role in thyroid hormone metabolism and bioavailability. Endocrine Reviews 22(4): 451-476.
17. Halestrap, A. P; and Price, N. T. 1999. The proton-linked monocarboxylate transporter (MCT) family: structure, function and regulation. Biochem.J. 343, 281-299.
18. Friesema, E; Docter, R; Moerings, E; Verrey, F; Krenning, E; Henneman, G; and Visser, Th. 2001. Thyroid hormone transport by the heterodimeric human system L amino acid transporter. Endocrinology 142(10): 4339-4348.
19. Kim, D. K, Kanai, Y., Chairoungdua, A., Matsuo, H., Cha, S. H,, and Endou, H. (2001) J. Biol. Chem. 276, 17221-17228
20. Kim, D. K., Kanai, Y., Matsuo, H., Kim, J. Y., Chairoungdua, A., Kobayashi, Y., Enomoto, A., Cha, S. H., Goya, T., and Endou, H. (2002) Genomics 79, 95-103
21. Mol, J. A., and Visser, T. J. (1985) Endocrinology 117, 1-7
22. Manning Fox, J. E., Meredith, D., and Halestrap, A. P. (2000) J. Physiol. 529, 285-293
23. Docter, R., Friesema, E. C., van Stralen, P. G., Krenning, E. P., Everts, M. E., Visser, T. J., and Hennemann, G. (1997) Endocrinology 138, 1841-1846
24. Wilson, M. C., Jackson, V. N., Heddle, C., Price, N. T., Pilegaard, H., Juel, C., Bonen, A., Montgomery, L, Hutter, O. F., and Halestrap, A. P. (1998) J. Biol. Chem. 273, 15920-15926.
25. Friesema, E. C., Docter, R, Krenning, E. P., Everts, M. E., Hennemann, G., and Visser, T. J. (1998) Endocrinology 139, 596-600

26. Jackson, V. N., Price, N. T., and Halestrap, A. P. (1995) Biochim. Biophys. Acta 1238, 193-196.

TABLE 1
Inhibition of uptake of T₄ and T₃ (10 nm) in MCT8
cRNA-injected oocytes by iodothyronines or Tyr, Trp and BSP.

% inhibition ± SEM

| Inhibitor | μM | T₄ | T₃ |
|---|---|---|---|
| L-T₄ | 10 | 69 ± 7 | 26 ± 7 |
| L-T₃ | 10 | 62 ± 7 | 57 ± 3 |
| D-T₄ | 10 | 76 ± 7 | 21 ± 5 |
| D-T₃ | 10 | 72 ± 3 | 24 ± 5 |
| Triac | 10 | 76 ± 3 | 47 ± 3 |
| BrAcT₃ | 10 | 90 ± 3 | 67 ± 1 |
| Tyr | 100 | 37 ± 6 | 0 ± 9 |
| Trp | 100 | 22 ± 6 | 0 ± 7 |
| BSP | 100 | 94 ± 2 | 81 ± 1 |

1. A method of altering transport of thyroid hormone or a functional part, derivative and/or analogue thereof across a membrane, said method comprising:
using a monocarboxylate transporter protein or a functional part, derivative and/or analogue thereof to alter transport across the membrane.

2. An isolated molecule capable of specifically binding a monocarboxylate transporter protein or a functional part, derivative and/or analogue thereof, or a ligand of said monocarboxylate transporter protein.

3. An isolated molecule capable of specifically binding a nucleic acid sequence encoding a monocarboxylate transporter protein or a functional part, derivative and/or analogue thereof, and/or encoding a ligand of said monocarboxylate transporter protein or functional part, derivative and/or analogue thereof.

4. A method of altering transport of a thyroid hormone or a functional part, derivative and/or analogue thereof across a membrane, comprising:
providing said membrane with the isolated molecule of claim 3 so as to alter transport of a thyroid hormone across the membrane.

5. The method according to claim 1, wherein said monocarboxylate transporter protein comprises monocarboxylate transporter protein-8.

6. The method according to claim 1, wherein the membrane comprises a plasma membrane.

7. The method according to claim 1, wherein said transport of thyroid hormone or functional part, derivative and/or analogue thereof is enhanced.

8. A composition comprising:
a compound capable of influencing the binding or transportation of a ligand of, or capable of binding to, a plasma membrane polypeptide capable of transporting a thyroid hormone, wherein said polypeptide comprises a monocarboxylate transporter protein or a functional part, derivative and/or analogue thereof.

9. The composition of claim 8 wherein said polypeptide is capable of transporting 3,3′,5-triiodothyronine.

10. The composition of claim 8 wherein said monocarboxylate transporter protein comprises monocarboxylate transporter protein-8.

11. A method of treating a subject suffering from a disease state selected from the group consisting of thyroid hormone-related disorder, non-thyroidal illness, obesity, cardiovascular illness or combinations thereof, the method comprising:
providing to the subject the pharmaceutical composition of claim 8.

12. A method for altering transport of a thyroid hormone or a functional part, derivative and/or analogue thereof across a membrane, said method comprising:
providing said membrane with
(a) a monocarboxylate transporter protein or a functional part, derivative and/or analogue thereof,
(b) a molecule capable of specifically binding a monocarboxylate transporter protein or a functional part, derivative and/or analogue thereof,
(c) a ligand of the monocarboxylate transporter protein,
(d) a molecule capable of specifically binding a nucleic acid sequence encoding a monocarboxylate transporter protein or a functional part, derivative and/or analogue thereof, and/or
(e) a molecule capable of specifically binding a nucleic acid sequence encoding a ligand of the monocarboxylate transporter protein or functional part, derivative and/or analogue.

13. A method for treating a disorder selected from the group consisting of thyroid metabolism, non-thyroidal illness, obesity, cardiovascular illness, and any combination thereof, said method comprising:
providing the composition of claim 8 to an individual suffering from the disorder.

14. A method for treating a subject suffering from a disorder selected from the group consisting of a thyroid hormone-related disorder, a disorder of thyroid metabolism, non-thyroidal illness, obesity, cardiovascular illness and any combination thereof, said method comprising:
providing to the subject at least a functional part of a monocarboxylate transporter protein, or a nucleic acid encoding at least a functional part of a monocarboxylate transporter protein, so as to treat the disorder.

15. The method according to claim 11, wherein providing comprises use of a gene delivery vehicle or gene therapy to provide.

16. A method for detecting and/or generating a compound having pharmacological activity, comprising:
screening a candidate compound with at least a functional part of a monocarboxylate transporter protein, or a nucleic acid encoding at least a functional part of a monocarboxylate transporter protein, in a method for detecting and/or generating a pharmaceutical compound.

17. The method according to claim 12, wherein said monocarboxylate transporter protein comprises monocarboxylate transporter protein-8.

18. A bioassay to identify or detect a candidate drug capable of binding to or influencing a plasma membrane a polypeptide comprising a monocarboxylate transporter protein or a functional part, derivative and/or analogue thereof, said bioassay comprising:
using, in said bioassay, a component selected from the group consisting of:
at least a functional part of a monocarboxylate transporter protein,
a nucleic acid encoding at least a functional part of a monocarboxylate transporter protein,
a molecule capable of specifically binding at least part of said monocarboxylate transporter protein or a ligand thereof,
a cell comprising encoding at least a functional part of a monocarboxylate transporter protein,
a cell comprising at least part of said monocarboxylate transporter protein, and any combination of any thereof.

19. The bioassay according to claim 18 wherein said monocarboxylate transporter protein comprises monocarboxylate transporter protein-8.

20. A composition comprising an isolated monocarboxylate transporter-8 protein or a functional part, derivative and/or analogue thereof.

21. The composition of claim 20 wherein said isolated monocarboxylate transporter-8 comprises: rat monocarboxylate transporter-8 protein.

22. A vector comprising a nucleic acid sequence encoding a monocarboxylate transporter-8 protein or a functional part, derivative and/or analogue thereof, and/or a molecule capable of specifically binding a monocarboxylate transporter-8 protein or a functional part, derivative and/or analogue thereof, or capable of specifically binding a ligand of said monocarboxylate transporter-8 protein.

23. A gene delivery vehicle comprising the vector of claim 23.

24. A method for determining whether an individual is suffering from, or is at risk of suffering from, a thyroid hormone-related disorder, non-thyroidal illness, obesity and/or cardiovascular illness, said method comprising:
determining whether the individual has a mutation in a nucleic acid sequence encoding a monocarboxylate transporter protein, and/or encoding a ligand of said monocarboxylate transporter protein.

25. The method according to claim 24, wherein said mutation comprises a deletion.

26. The method according to claim 24, wherein mutation comprises a deletion of at least one exon.

27. The method according to claims 25, wherein said monocarboxylate transporter protein comprises monocarboxylate transporter protein-8.

28. A diagnostic kit comprising:
suitable means for detecting a mutation in a nucleic acid sequence encoding a monocarboxylate transporter protein or a functional part, derivative and/or analogue thereof, and/or encoding a ligand of said monocarboxylate transporter protein or functional part, derivative and/or analogue.
